(19) United States
(12) Patent Application Publication
(10) Pub. No.: US 2009/0143650 A1
(43) Pub. Date: Jun. 4, 2009

(54) MINIATURIZED, DERMAL-ADHESIVE-BASED DEVICE FOR POSITION-INDEPENDENT, NON-INVASIVE FETAL MONITORING

(75) Inventors: Marie A. Guion-Johnson, Farmington, MN (US); David Boudreault, Palo Alto, CA (US); Beverly Tang, Palo Alto, CA (US); Shivinand Lad, Palo Alto, CA (US); Brian Fahey, Palo Alto, CA (US); Jeremy Collins, Saratoga, CA (US); Elizabeth Langen, Stanford, CA (US)

Correspondence Address: SHUMAKER & SIEFFERT, P. A., 1625 RADIO DRIVE, SUITE 300, WOODBURY, MN, 55125, US

(73) Assignee: Regents of the University of Minnesota

(21) Appl. No.: 12/274,889

(22) Filed: Nov. 20, 2008

Related U.S. Application Data
(60) Provisional application No. 61/004,482, filed on Nov. 28, 2007.

(51) Int. Cl. A61B 5/00; A61B 5/02

(52) U.S. Cl. 600/301; 600/528

(57) ABSTRACT

A system for fetal position-independent, non-invasive fetal monitoring includes a plurality of disposable adhesive patches for placement on an expectant mother's upper and lower abdomen. Each of the patches includes one or more miniature electronic devices embedded within the adhesive patches to detect: (i) heart sounds of a fetus within the mother, (ii) heart sounds of the mother, and (iii) signals indicative of uterine contractions of the mother. A processing hub having a receiver to receive signals from the plurality of patches, wherein the processing hub receives and processes primary signal from the primary patch and the secondary signals from the secondary patches to triangulate the location of the fetus, cancel noise in the primary signal and increase the amplitude of the primary signal for more reliable reporting.

This application claims the benefit of U.S. Provisional Patent Application 61/004,482, entitled “A MINIATURIZED, DERMAL-ADHESIVE-BASED DEVICE FOR POSITION-INDEPENDENT, NON-INVASIVE FETAL MONITORING,” filed Nov. 26, 2007, the entire content of which is incorporated herein by reference.

### BACKGROUND

Current fetal monitoring includes a device to monitor fetal heart rate and a second device to monitor uterine contractions. The fetal heart rate monitor is an ultrasound device that is placed on the mother's abdomen closest to the baby's heart. The tocodynamometer (TDM) measures uterine contractions through a pressure disc placed in the upper abdomen. The TDM does not give absolute strength of contractions, but does allow for relative comparisons and timing with changes in fetal heart rate. These two devices are connected to a monitor that displays a continuous readout.

There are a number of limitations to the currently used EFM systems. At present, EFM is time consuming to set up, signal strength is affected by patient's position, carries a high false positive rate, requires continued manipulation and adjustment by nursing and requires straps to secure the devices that are not only uncomfortable to the patient, but limit mobility and interfere with fetal monitoring during placement of an epidural.

The following situations or conditions may result in fetal distress:
Cord compression (there is no free blood flow to the fetus),
Fetal heart block (where there is a block of electrical flow within the heart muscle causing an altered heart rhythm),
Fetal malposition,
Fetal hypoxia (insufficient oxygen supply to the fetus),
Infection (monitoring cannot diagnose an infection, but can suggest the presence of an infection),
Uteroplacental insufficiency (insufficient oxygen exchange between the uterus and the placenta),
Fetal distress, and
Abruptio placenta.

### SUMMARY

This device is designed to provide comfortable and reliable external fetal monitoring during the labor and delivery process. While the current form of the device is intended for a hospital setting, future generations of the device could extend it to antepartum clinic and home use, leveraging technology platforms to make fetal monitoring widely accessible to pregnant mothers via personal computers or handheld devices.

In one embodiment, this device comprises a plurality of disposable adhesive patches placed on the expectant mother's upper and lower abdomen. Thin-film technologies are employed to embed miniature devices to detect fetal and maternal heart rate and uterine contractions and associated electronic equipment into a dermal adhesive. In addition, each patch contains a data transmission mechanism that allows these signals to be sent to a post-processing hub at the mother's bedside. This bedside computer can then be linked to base stations at the nurses' desk to allow for monitoring of more than one laboring mother at a time (FIG. 1).

This device hones in on the desired signals, independent of fetal or uterine position. The setup of the device facilitates the reporting of maternal heart rate, fetal heart rate, and uterine contraction from the same or different patches, depending on the strength of those signals at that particular patch location. Furthermore, signals from the secondary patches are post-processed to triangulate the location of the fetus, cancel noise and increase the amplitude of the primary signal for more reliable reporting, and easily distinguish between fetuses in twin or triplet gestations.

Therefore, once the patches are placed, they no longer need to be adjusted by the nursing, obstetrics, or anesthesia staff. Typically, the patches do not interfere with common obstetric procedures including but not limited to cesarean sections and epidurals allowing uninterrupted monitoring during these procedures. In addition, use of wireless technology allows the mother freedom to move around and change position without becoming uncomfortably hindered or entangled.

This device provides a more comfortable, reliable, continuous, and manageable way to monitor fetal and maternal vital signs during labor. The system uses miniaturization and advanced signal processing algorithms to provide familiar, but more dependable, monitoring to physicians and nurses.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 which illustrates the device and system and its components according to a preferred embodiment of the invention.

FIG. 2 is a flowchart illustrating showing signal processing for separation of the three outputs: fetal heart rate, maternal heart rate and uterine contractions.

### DETAILED DESCRIPTION

This invention provides a device, system and method for performing continuous and non-invasive monitoring of fetal heart rate and maternal contractions. This works through a system designed to be affixed to a pregnant woman's abdomen and the acquired signal and/or analyzed data is transmitted to a central unit where the medical staff can quickly respond to fetal distress.

In the following description, various aspects of the present invention will be described. For purposes of explanation, specific configurations and details are set forth in order to provide a thorough understanding of a preferred embodiment of the present invention. However, it will also be apparent to one skilled in the art that the present invention may be practiced without the specific details presented herein. Furthermore, well-known features may be omitted or simplified in order not to obscure the present invention.

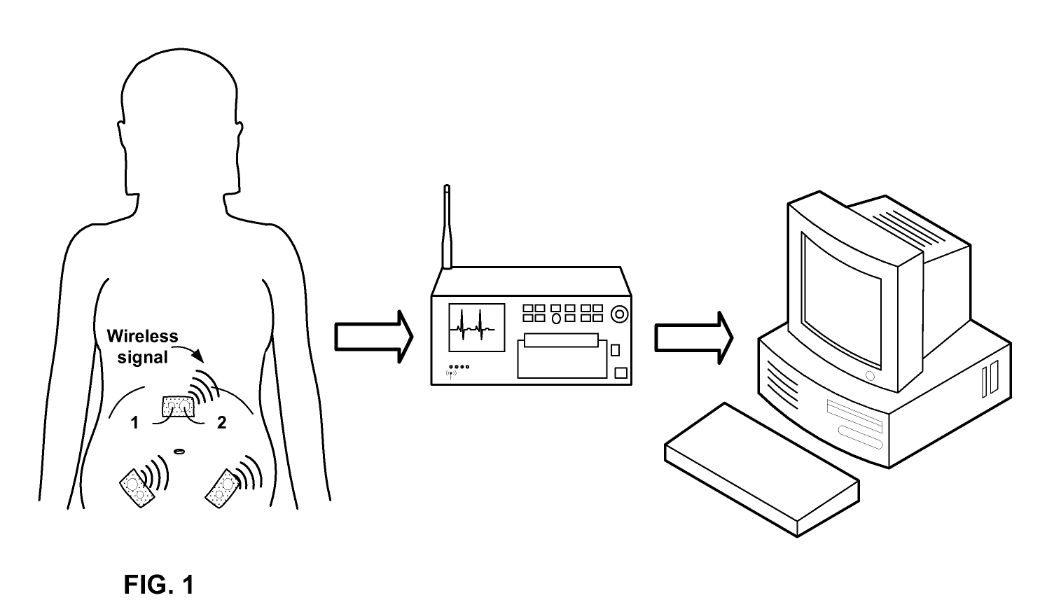

Reference is now made to FIG. 1 which illustrates the device and system and its components according to a preferred embodiment of the invention. FIG. 1 illustrates a fetal monitoring device. In this one embodiment of the device, three identically-equipped patches each with 2 unique sensors (labeled 1 and 2), are placed on the laboring mother's abdomen to detect heart rate and uterine contractions. These sensors output signals wirelessly to a post-processing hub at the mother's bedside that incorporates the familiar user interface of current fetal monitoring devices. Signals can be displayed on a screen, saved to a database, and printed on thermal paper. The bedside computer is linked to a central computer at the nurses' station to accommodate the monitoring of multiple laboring mothers in different rooms

In a preferred embodiment of the present invention, the device and system is typically comprised of 3 patches that each contain two unique sensors, 1 and 2.

It will be appreciated by those skilled in the art that a plurality of sensors may be used either independently or in a variety of combinations in the device and system of the invention in order to accurately acquire the fetal heart rate. These sensors may refer to any element suitable for sensing the presence of heart rate (maternal and fetal) and maternal uterine contractions. Examples of ways that this device might use to detect heart rate include, but are not limited to: Electrocardiography, auscultation, Doppler, visual, angiogram, echocardiography, functional magnetic imaging, fluoroscopy, arterial line, pulse oximetry, palpation of pulse, etc. Examples of ways that this device might use to detect uterine contractions include, but are not limited to: strain, electomyogram, ultrasound, accelerometer, rigidity, tension, gyroscope, sound, vibration, visual, pressure, etc.

In an embodiment of the present invention, the device comprises three disposable adhesive patches placed on the expectant mother's upper and lower abdomen. Thin-film technologies are employed to embed miniature auscultation devices (microphones), piezoelectric sensing crystals, and associated electronic systems into a hypoallergenic, breathable dermal adhesive. Each patch can be equipped with identical components that allow for the monitoring of fetal and maternal heart rate (thin-film auscultation apparatus) and uterine contractions (thin-film piezoelectric apparatus). Examples of surface materials that may be used in the present embodiment may include but are not limited to: quartz, gallium, orthophosphate, langosite, polyvinyl, fluoride.

In an alternative embodiment, the adhesive patches would be disposable, while the sensors would be reusable. In this design, each of the patches will have an integrated docking site including electrical connections for electronic sensors 1 and 2, along with a transmitter to send received data to a bedside unit. These sensors attach to the disposable patch through a reversible docking station. Alternatively, the entire sensing device including the adhesive patches and the sensors may be disposable.

Additionally, the bedside unit would display continuous data plotted for easy viewing and interpretation. This unit could contain the processor for interpreting the data displayed and then transmitting to a central area for monitoring multiple laboring patients. Alternatively, this bedside unit could simply act as a relay station sending its data to a central processor that would analyze multiple signals from multiple laboring patients. From the central processor, the bedside unit would receive its processed data for display at the bedside.

### Fetal Heart Rate Detection

In one incarnation of the invention, three patches containing sensor-type 1 are placed in a standard fashion shown in FIG. 1. The signal received from the separate patches could be transmitted for processing where it would be identified, filtered and amplified to obtain an isolated fetal heart rate. Using multiple sensors would also allow for the multilateration of the heart rate signal location. This would also allow for a position independent method for obtaining the fetal heart rate. The processing could either be at the bedside unit or at the central monitoring unit as described above. Examples of possible techniques that this device may use to detect heart rate may include, but are not limited to the following: Electrocardiography, auscultation, Doppler, visual, angiogram, echocardiography, functional magnetic imaging, fluoroscopy, arterial line, pulse oximetry, palpation of pulse, etc. The heart rates could be displayed at the bedside unit in parallel with uterine contractions. The bedside unit could then communicate with the central monitoring station. The monitoring unit could have alarms to alert the medical staff of fetal distress to optimize early intervention.

### Uterine Contraction Detector

In one incarnation of the invention, patches containing sensor 2 would be placed in an distribution shown in FIG. 1. The auscultatory sounds received simultaneously from the separate patches are detected and transmitted for processing where the signals would be combined to identify uterine contractions and show their temporal relation to the fetal heart rate. Given that the location to detect maximal effect of contraction varies, using multiple sensors would allow for summation of the signals and position-independent contraction monitoring. Examples of ways that this device might use to detect uterine contractions include, but are not limited to: strain, electomyogram, ultrasound, accelerometer, rigidity, tension, gyroscope, sound, vibration, visual, pressure, and the like.

One embodiment of the invention would use a mechanical-electrical transducer to sense contractions. These sensors would pick up subtle changes in tension which would provide information about the duration of contractions and its timing with fetal heart rate. This would be displayed in parallel with the fetal heart rate on the bedside unit.

Another variation would be to combine a patient input button that would allow the machine to be trained early in the process, before an epidural is placed. When the patient feels a contraction coming on, she presses a button. When the patient perceives the contraction has ended, she again presses the same button. This allows the machine to cross reference its signal processing with real patient input. The transducer that most closely correlates with the patients contraction input is then selected as the primary detector. The remaining transducers can then add to the primary detectors signal recording, when it detects a signal. Alternatively, the other two sensors can be silenced to conserve power.

Other methods to acquire this signal using mechanical-electric transducers include, but not limited to: Constant recording using all three detectors, only detecting uterine contractions when the fetal heart tracing is outside normal variation, constant patient input regarding perceived contractions, etc.

### Signal Processing

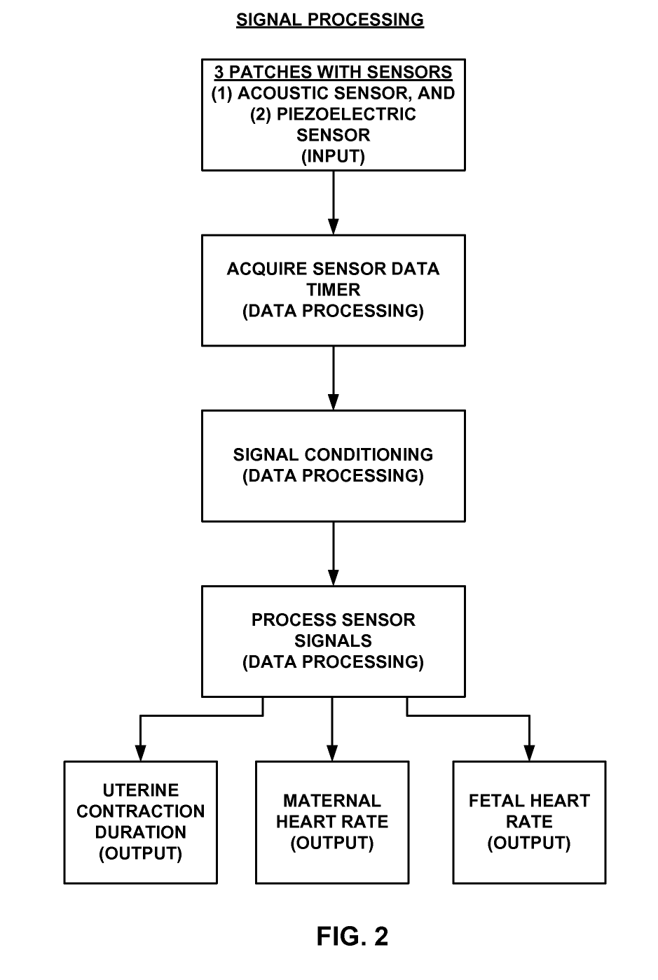

In one incarnation of the invention, patches containing both or either sensor 1 and 2 would be placed on the laboring patient's abdomen. The signals received from the sensors could be processed as outlined in FIG. 2, which is a flowchart showing signal processing for separation of the three outputs: fetal heart rate, maternal heart rate and uterine contractions.

Signal conditioning may include, but is not limited to, filtering, amplification, upsampling, downsampling, and other operations. Types of filters that may be incorporated include, but are not limited to, band pass filters, time of flight cancellation, blind source separation, and the like.

Processing of sensor signals may include, but is not limited to, Fourier transformation, wavelets, principal components analysis, singular value decomposition, hidden Markov models, other statistical analysis techniques, and the like.

### Signal Transmission

One incarnation of this invention's signal transmission is a wireless signal from each patch to the bedside unit and from the bedside unit to the central monitoring station. This would allow the patient to ambulate while being monitored and facilitate procedures such as epidurals. The invention would allow for continuous monitoring during transportation and throughout delivery process (vaginal or cesarean section). The methods for wireless transmission include, but are not limited to: WiFi, Bluetooth, Infrared, Radio Frequency, acoustic, optical, laser, Morse code, etc. The device could also be connected using wires from the patch to the bedside unit and wireless from the bedside unit to the central monitoring station. Alternatively, it could be wireless from the patch to the bedside unit and wired from the bedside unit to the central monitoring station. Finally, the device could be connected completely using wires.

In the preferred incarnation of the invention, the patch would contain Bluetooth wireless technology to transmit a signal to the bedside unit, which would then transmit the processed signal to the central monitoring station via WiFi or hospital internet.

### Signal Display

One embodiment of the current invention's display includes, but is not limited to an interactive screen that allows the user to touch-navigate the menu of options. When not touched the display would show post-processing tracings of both the fetal heart rate and uterine contractions. This same information would be transmitted to the central processing station where an identical view can be seen. The bedside unit would also allow for printing of either segments of concerning rhythms or a complete record of tracings for medical records. The menu options would include, but would not be limited to all necessary display adjustments, print options, help menu, setup instructions, alarms, etc.

A possible display option may include a three dimensional representation of signals received, giving the user visual feedback on the sounds relative location.

An alternative embodiment employs the use of a non-interactive display screen (including, but not limited to, LCD, TFT, plasma, CRT, etc.) with user input buttons and/or dials to the side.

### Power Source

The power source for the above described invention will vary based on the component needing power. In general the power can be supplied by a number of suitable power sources. For example, but not be limited to the following: one or more batteries, rechargeable batteries, high density chemical batteries, high efficiency micro-batteries, removable batteries, electrochemical cells, fuel cells, solar-powered cells, or any other suitable electrical power source.

1. Patch

One incarnation of the current invention's patch power source would have a single use battery that is appropriately sized and of sufficient power to obtain and transmit its wireless signal. They would also allow for continuous monitoring. Alternatively, the patch can have rechargeable batteries that can be easily switched with batteries being charged at the bedside unit. The patch would become active when contact is made with the patient's skin. Alternatively the patch could have a switch or button to turn on the wireless technology. Another alternative embodiment is to have the patches activated by the base unit.

2. Bedside Unit

One incarnation of the present invention's bedside unit power source is to use a rechargeable battery that can be charged by plugging the unit to an alternating current outlet. The battery pack can also be swapped for those that are charged in a separate location. During transportation, the unit can be unplugged while continuing to receive and process signals. The unit would have a button, switch or any other mechanical way of quickly powering on and off the unit. The bedside unit could also charge the batteries which may be used interchangeable in the patches. Another embodiment of the bedside unit power supply would be completely powered by an alternating current outlet.

### Method of Use

In a preferred embodiment of the present invention, the device and system described above is intended to be integrated into the care of laboring women monitored in the hospital. The decision tree to use this technology would be identical to that for current external fetal monitoring and up to the discretion of the medical staff. Once the decision is made to monitor the patient, the bedside unit and patches are brought in to the room with the patient. The bedside unit is powered on and immediately asks for the patients name and medical record. Subsequently, the unit instructs the staff on the correct sequence for setting up the device. The bedside unit receives the signal from the separate patches and immediately starts processing the data. The processed signals are displayed on the bedside unit and sent to the central station.

While the current form of the device is intended for a hospital setting, future generations of the device could extend it to antepartum clinic or home use, leveraging existing technology platforms to make fetal monitoring widely accessible to pregnant mothers via personal computers or handheld devices.

Various embodiments of the invention have been described. These and other embodiments are within the scope of the following claims.

1. A device for fetal position-independent, non-invasive fetal monitoring comprising:
a plurality of disposable adhesive patches for placement on an expectant mother's upper and lower abdomen;
one or more miniature electronic devices embedded within the adhesive patches operable as an detector to detect: (i) heart sounds of a fetus within the mother, (ii) heart sounds of the mother, and (iii) signals indicative of uterine contractions of the mother.

2. The device of claim 1, wherein each of the plurality of disposable patches comprises two different types of sensors including: (i) a first sensor to detect the hearts sounds of the fetus and the mother, and (2) a second sensor to detect the uterine contractions.

3. The device of claim 2,
wherein the first sensor of each of the patches is an acoustic sensor for detecting the heart sounds for both the fetus and the mother, and
wherein the second sensor of each of the patches is a piezoelectric sensor for sensing the uterine contractions.

4. The device of claim 1, wherein the device outputs signals indicative of a maternal heart rate, a fetal heart rate, and the uterine contractions from the patches.

5. The device of claim 1, wherein the device one or more of the patches to be used for reporting depending on a strength of signals at each particular patch location for the maternal heart sounds, the fetal heart sounds and the uterine contractions.

6. The device of claim 1, wherein each patch contains a data transmission mechanism that allows signals to be wirelessly sent to a post-processing device.

7. The device of claim 1, wherein the adhesive patches are disposable and the embedded electronic detectors are removable from the patches and reusable.

8. The device of claim 7, wherein the removable electronic detectors electrically couple to the disposable adhesive patches by a docking station integrated within the patches and having electronic connections for the removable electronic detectors.

9. A system for fetal position-independent, non-invasive fetal monitoring comprising:
a plurality of disposable adhesive patches for placement on an expectant mother's upper and lower abdomen, wherein each of the patches includes one or more miniature electronic devices embedded within the adhesive patches to detect: (i) heart sounds of a fetus within the mother, (ii) heart sounds of the mother, and (iii) signals indicative of uterine contractions of the mother, and
a processing hub having a receiver to receive signals from the plurality of patches, wherein the processing hub receives and processes primary signal from the primary patch and the secondary signals from the secondary patches to triangulate the location of the fetus, cancel noise in the primary signal and increase the amplitude of the primary signal for more reliable reporting.

10. The system of claim 9, wherein each patch contains a data transmission mechanism that allows signals to be wirelessly sent to a post-processing device.

11. A method for performing continuous and non-invasive monitoring of fetal heart rate and maternal contractions, the method comprising:
affixing a plurality of a plurality of disposable adhesive patches on an expectant mother's upper and lower abdomen, wherein each of the patches include one or more miniature electronic devices embedded within the adhesive patches to detect: (i) heart sounds of a fetus within the mother, (ii) heart sounds of the mother, and (iii) signals indicative of uterine contractions of the mother; and
a processing hub having a receiver to receive signals from the plurality of patches, wherein the processing hub receives and processes primary signal from the primary patch and the secondary signals from the secondary patches to triangulate the location of the fetus, cancel noise in the primary signal and increase the amplitude of the primary signal for more reliable reporting.
